(19) United States
(12) Patent Application Publication
(10) Pub. No.: US 2009/0305228 A1
(43) Pub. Date: Dec. 10, 2009

(54) Potentiation of Apoptosis by Monoclonal Antibodies

(76) Inventors: Nathalie Fournier, Marcq En Baroeul (FR); Christophe De Romeuf, Lambersart (FR)

Correspondence Address: Leyendecker & Lemire, LLC, C/O PORTFOLIO IP, P.O. BOX 52050, MINNEAPOLIS, MN, 55402, US

(21) Appl. No.: 12/097,686

(22) PCT Filed: Dec. 15, 2006
(86) PCT No.: PCT/FR06/02767

§ 371 Date: Sep. 24, 2008

(30) Foreign Application Priority Data
Dec. 16, 2005 (FR) 0512815

(51) Int. Cl. C12Q 1/70; C12N 5/06; G01N 33/53

(52) U.S. Cl. 435/5; 435/375; 435/349; 435/7.21

(57) ABSTRACT

The present invention relates to the use of an antibodies composition, the fucose content of which is less than 65%, for indicating apoptosis in vitro.

The present invention relates to the use of an antibody composition whose fucose content is less than 65%, for in vitro apoptosis induction.

Increasingly used in research, antibodies also form tools of choice for diagnosis and therapy in which they provide an alternative to conventional treatments.

Numerous antibodies having therapeutic use, of plasma or monoclonal origin, are currently on the market or under clinical development. Their properties are used to obtain therapeutic tools capable of specifically binding to their target, and of efficiently recruiting immune effector cells, thereby causing the destruction of the target cell by means of the cytotoxic functions of these cells.

However, in some pathologies, such as chronic lymphocytic leukaemia (CLL) patients are observed to have deficient activity of immune effector cells, in particular of NK cells, which may lie at the cause of an increased incidence of secondary pathologies in these patients (Ziegler et al, 1981). Also, when these patients are treated for their CLL with the anti-CD20 monoclonal antibody Rituxan, this treatment appears to have little efficacy which could be partly accounted for by the low ADCC activity of their NK cells (Farag 2003).

NK cell deficiency is also found in patients suffering from lupus erythematosus (Green 2005), patients with dilated cardiomyopathy (Anderson 1982), patients suffering from histiocytosis with haemophagocytosis (perforin deficiency—Clementi R, 2005) and HIV-infected patients (perforin and granzyme deficiency—Portales, 2003). It can therefore be feared by analogy to the foregoing that, for CLL patients, treatments with conventional monoclonal antibodies will have little or no efficacy.

The Applicant has therefore sought to provide tools whose activity is independent of the cytotoxic functions of immune effector cells, and which can therefore be given to patients having reduced activity of immune effector cells.

In document WO 01/77181 the Applicant demonstrated the importance of selecting cell lines which can be used to produce antibodies having strong ADCC activity via the FcgammaRIII receptor (CD16). It was shown therein that modified glycosylation of the Fc region in antibodies produced in rat myeloma lines such as YB2/0 led to improving ADCC activity. The glycan structures of said antibody compositions impart low fucosylation to the antibody composition.

In surprising manner, it has been found by the Applicant that said antibody compositions, in addition to their strong cytotoxic activity, and in the presence of cells expressing CD16 on their surface, induce substantial apoptosis, whereas the same antibody produced in CHO potentiates apoptosis to a much lesser extent.

### DESCRIPTION

The invention therefore relates to the use of an antibody composition whose fucose content is less than 65%, for inducing apoptosis in vitro, ex vivo or in vivo.

A first object of the invention relates in particular to a method to induce in vitro, ex vivo or in vivo apoptosis of a target cell using an antibody composition, which comprises contacting the antibody composition with the target cell in the presence of cells expressing CD16 on their surface, the antibodies being directed against the target cell and the antibody composition having a fucose content of less than 65%.

The antibodies consist of heavy chains and light chains, bound together by disulfide bridges. Each chain, at N-terminal position, consists of a variable region (or domain) specific to the antigen against which the antibody is directed and, at C-terminal position, of a constant region consisting of a single CL domain for light chains and of several domains (CH₁, CH₂ and CH₃) for heavy chains. The association of the variable domains and CH₁ and CL domains of the heavy and light chains forms the Fab fragments of the antibody, which are connected to the Fc region by a very flexible hinge region enabling each Fab to bind to the target antigen. The Fc region, which mediates the effector properties of the antibody, remains accessible to the effector molecules such as the FcγR receptors (FcgammaR). The Fc region, consisting of 2 globular domains CH₂ and CH₃ is glycosylated at the CH₂ domain with the presence on each of the 2 chains, of a bi-antenna N-glycan bound to asparagine 297 (Asn 297).

Said N-glycann is in the following general form (shown form: “G0”) to which other sugars may be added:

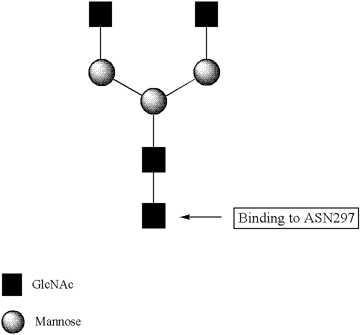

Therefore in the antibody composition of the invention, by “fucose” is meant the fucose carried by these N-oligosaccharides. The fucose molecule, when present, is bound to the N-acetylglucosamine (GlcNAc) of the N-oligosaccharide, this GlcNAc itself being bound to Asn 297.

Each of the N-glycans carried by either of the 2 heavy chains of each antibody may or may not carry a fucose molecule. Therefore each antibody may comprise 0, 1 or 2 fucose molecules, according to whether none of its N-glycans carries fucose, only one of its N-glycans carries a fucose molecule, or its 2 N-glycans each carry a fucose molecule.

Therefore, by “antibody composition whose fucose content is lower than 65%” is meant an antibody composition in which, among all the glycan structures carried by each glycosylation site (Asn 297) of the antibodies of the composition, less than 65% comprise a fucose molecule.

Advantageously, said glycan structures are more particularly chosen from among the following forms:

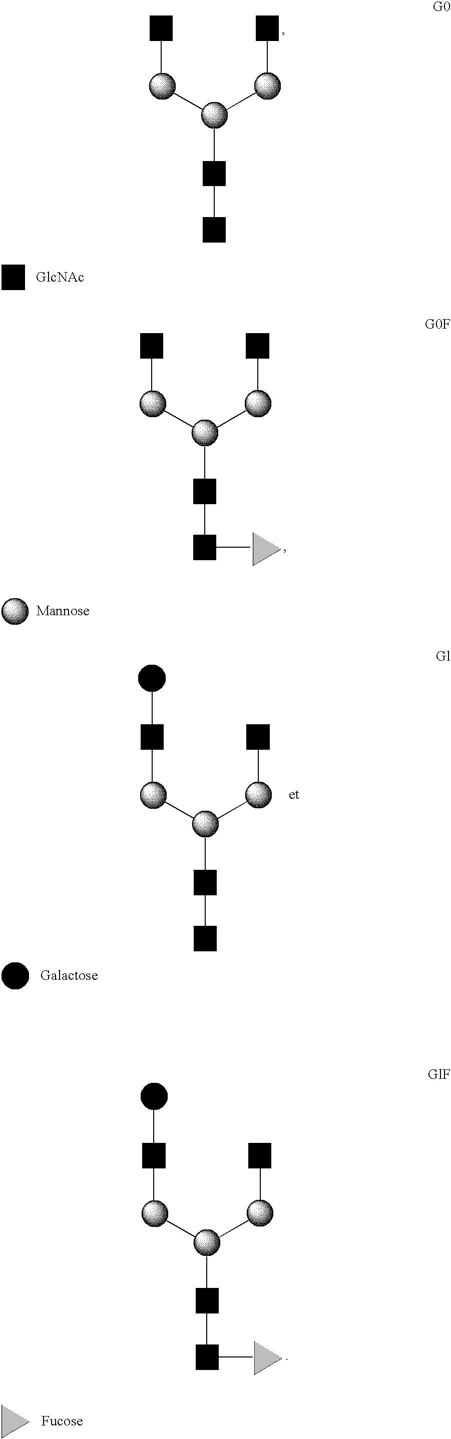

Therefore, advantageously, amongst the glycan structures of form G0, G0F, G1 and G1F carried by each glycosylation site (Asn297) of the antibodies of the composition according to the invention, the fucose content is less than 65%.

Said antibody composition, and its advantageous characteristics regarding the ADCC induction (Antibody-Dependent Cell-mediated Cytotoxicity) are described in document WO 01/77181.

The antibody compositions of the invention have the particularity and the advantage of triggering a strong apoptosis reaction. Apoptosis plays a major role in triggering cell death. Numerous factors are involved in apoptosis induction, but they all arrive at a common pathway passing through the mitochondria, protein Bcl-2 and the caspases.

The chief mechanisms initiating programmed cell death are stress, (e.g. hypo-oxygenation), treatment with cytotoxic substances or corticoids, deprivation of growth factors, DNA damage and the transmission of a death signal (Fas receptor of cytotoxic lymphocytes and of natural killers, of TNF-α necrosis factor).

Subsequent to a cell death signal, the caspases are activated causing activation of the proteins associated with apoptosis. The phosphatidylserines are then translocated from the inner surface of the cell membrane to the outer surface. They can be visualized in vitro by the fixing of Annexin V to the cell surface. Condensation of the cytoplasm, core and chromatin, DNA fragmentation, blebbing of the plasma membrane and loss of membrane asymmetry then become visible. At this stage, the cells fix propidium iodide in vitro (DNA intercalator agent). Apoptotic bodies are then formed which are digested by the surrounding macrophages.

Potentiation of the apoptosis induced by the antibody compositions of the invention is not due to the cytotoxic functions of the immune effector cell, but is induced by a strong clustering of the Fc region of the antibodies with the CD16 receptor expressed on the surface of the immune effector cells.

Advantageously, the antibody composition of the invention has a content of more than 60%, preferably more than 80% of G0+G1+G0F+G1F forms, on the understanding that the content of G0F+G1F forms is less than 50%, preferably less than 30%.

In vitro demonstration of apoptosis induction by the antibody compositions of the invention was conducted with CD16 transfected Jurkat cells devoid of cytolytic activity, contrary to NK cells. This forms a model in which cell death can only be induced by apoptosis.

Therefore, the method of the invention, intended to induce in vitro apoptosis of a target cell, is carried out by means of cells expressing CD16 on their surface, but which are not effector cells. These cells therefore do not have or no longer have cytotoxic activity against target cells. For example, the CD16 Jurkat cell can be mentioned.

In vivo potentiation of apoptosis by the compositions of the invention is obtained with any effector cells expressing CD16, in particular peripheral blood cells expressing CD16, such as monocyte-macrophages, neutrophils, NK cells and certain T-cell sub-populations. Therefore the compositions of the invention can be used in the presence of quantitative or qualitative deficiency (no ADCC) of a population of immune effector cells (cytotoxic cells). In this case, this potentiation is possible through other cells expressing CD16 on their surface (CD16+ cells), in particular cells expressing CD16 on their surface but which do not have or no longer have any cytotoxic activity.

Said deficiencies are found in patients with B-cell CLL (chronic lymphocytic leukaemia) in whom a drop in activity of NK cells is observed (Foa 1986, Ziegler 1981) or more specifically a deficiency of cytolytic molecules (Katrinakis 1996), and patients suffering from lupus erythematosus in whom NK cell deficiency has been described (Green 2005). The antibody compositions can be used according to the invention in said pathologies.

Advantageously, the antibody composition of the invention has a fucose content of less than 55%, or even less than 40%, 30% or 20%.

In particular advantageous manner, the fucose content of the antibody composition lies between 20% and 45%, or between 25% and 40%.

In particularly advantageous manner, the antibody composition of the invention also has a fucose content/galactose content ratio of less than 0.6. Advantageously, this ratio is less than 0.5; less than 0.4; less than 0.2 or further advantageously less than 0.1. For the purposes of the present invention, by “fucose content/galactose content ratio” is meant the ratio between the fucose content of the antibody composition and the galactose content of the antibody composition, these 2 sugars able to be carried by each glycan structure bound to each glycosylation site (Asn 297) of the antibodies of the composition.

Advantageously, the antibody composition used in the invention is produced by the YB2/0 cell line, or any cell derived from YB2/0 and imparting the same characteristics as regards post-translational modifications of the proteins, in particular regarding glycosylation.

This line was chosen on account of its capacity to produce antibody compositions of which some, after selection, show low fucosylation, the usefulness of this property being demonstrated by the Applicant in the present invention for the production of antibody compositions capable of strong apoptosis induction.

Therefore a further object of the invention is the use of the YB2/0 cell line to produce antibodies with strong apoptosis capability.

A further object of the invention is a method to select antibody compositions with strong apoptosis capability comprising the following steps:
1) contacting the antibody to be evaluated with the antigen of said antibody (or a cell expressing this antigen) in the presence of cells expressing the CD 16 receptor on their surface,
2) measuring induced apoptosis,
3) selecting antibody compositions showing apoptosis induction greater by at least 20%, even greater than 50% or more, in the presence of the cell expressing CD 16, compared with the negative control.

When carrying out step 1) of this method, the negative control can be obtained for example in the presence of cells not expressing CD 16.

For step 2), measurement of induced apoptosis can be made in several manners, using specific conventional techniques to measure apoptosis such as the measurement of Annexin V (early stage of apoptosis measuring the phosphatidylserines exposed on the cell surface) associated with propidium iodide (advanced stage of apoptosis measuring DNA degradation) or other markers such as YO Pro-1 (DNA intercalator agent measuring the fragmentation of this DNA).

For the purpose of the invention, by “strong apoptosis capability” is meant a capacity to induce apoptosis that is greater by at least 20%, even greater by 50% or more, in the presence of the CD16-expressing cell, compared with the negative control.

To implement this method, the cells expressing CD16 on their surface can be CD16-transfected cells, in particular Jurkat cells, cells of peripheral blood expressing CD16 e.g. monocyte-macrophages, neutrophils, NK cells and some subpopulations of T-cells, this list not being restrictive. Preferably, the cells expressing CD16 on their surface are cells which do not have or no longer have any cytotoxic activity.

Advantageously, the method of the invention also comprises a step of determining the fucose content of the chosen antibody compositions. This determination can be performed before the step in which the antibody to be evaluated is contacted with the antigen, or after the selection of antibody compositions showing induced apoptosis that is greater than that of the negative control.

If the determination of the fucose content of the antibody composition is performed before the step to contact the antibody to be evaluated with the antigen, it is advantageous to choose antibody compositions having a fucose content of less than 65%. Preferably, the chosen antibody compositions have a fucose content of less than 30% or between 20% and 45%, or between 25% and 40%.

Advantageously, the chosen antibodies are anti-idiotype antibodies to anti-factor VIII inhibitors, antibodies directed against auto-antibodies to target memory B lymphocytes, and antibodies directed against viral and/or bacterial proteins expressed on the surface of infected cells, for the purpose of inducing their elimination. It is also possible to select any antibody recognizing an antigen expressed preferably on the surface of tumour cells, including haematopoietic proliferations.

A further object of the invention relates to the use of monoclonal antibody compositions whose fucose content is less than 65%, preferably less than 30%, further preferably between 20% and 45% or between 25% and 40%, or antibody compositions selected using the above-described selection method, to prepare a medicinal product which, without involving the cytotoxic functions of immune effector cells, is intended to treat quantitative or qualitative NK-cell deficiency (no ADCC) chosen in particular from among B-CLL (B-cell chronic lymphocytic leukaemia), lupus erythematosus, dilated cardiomyopathy, histiocytosis with haemophagocytosis, as well as HIV and haemophilia A or B.

Said antibody compositions show interaction with CD16 that is at least 2 times greater, even advantageously 10 times greater than the therapeutic antibody Rituxan.

The interaction between the Fe region of the antibody and the CD16 receptor can be measured via direct or indirect binding by competition with an anti-CD16 monoclonal antibody of 3G8 type directed against the binding site of the Fc part of the antibodies, on CD16 positive cells.

Advantageously, the antibody composition used according to the invention has a content of more than 60%, preferably more than 80% of G0+G1+G0F+G1F forms, it being understood that the content of forms G0F+G1F is less than 50%, preferably less than 30%.

In particularly advantageous manner, the antibody composition used according to the invention also has a ratio of fucose content/galactose content of less than 0.6. Advantageously this ratio is less than 0.5; less than 0.4; less than 0.2 or further advantageously less than 0.1.

Advantageously, this use is intended for the preparation of a medicament to treat pathologies in which one or more effector cell populations is reduced or even null, said cells therefore inducing low cytotoxic activity via ADCC.

A further object of the invention relates to the use of anti-CD20 monoclonal antibodies having strong interaction with CD 16, selected according to the method of the invention, or meeting the above-cited fucosylation characteristics, to manufacture a medicament which, without involving the cytotoxic functions of the immune effector cells, is intended to treat patients suffering from B-CLL and in whom a decrease in the lysis functions of NK cells may be observed.

Generally the antibody of the invention can be used to treat any haematopoietic proliferation.

A further object of the invention is the use of said monoclonal antibody compositions to manufacture a medicament which, without involving the cytotoxic functions of the immune effector cells, is intended to treat patient having NK cell deficiency, and who cannot be treated by antibodies whose cytotoxicity is expressed solely by means of the NKs.

By way of example, mention may be made of patients suffering from lupus erythematosus (Green 2005), patients with dilated cardiomyopathy (Anderson 1982), patients suffering from histiocytosis with haemophagocytosis (perform deficiency) (Clementi R, 2005) and HIV-infected patients (perforin and granzyme deficiency) (Portales, 2003).

Those persons can also be included who suffer from trisomy-21 (Nurmi 1982) and patients receiving grafts in whom associated treatments lead to NK cell deficiency and an increased risk of developing tumours (Alamartine 1997). In haematopoietic pathologies, in addition to B-CLL patients, a deficiency in cytotoxicity related to NK cells has been described in patients suffering from acute myelogenous leukaemia (Nasrallah, 1983) and hairy cell leukaemia (Trentin 1990).

More generally, smoker populations (Takeuchi 2001) show reduced NK activity compared with non-smokers. Additionally, the antibodies according to the invention can be particularly advantageous for the treatment of patients suffering from haemophilia A or B, optionally to target memory B lymphocytes.

Other aspects and advantages of the invention are described in the following examples which are to be construed as illustrative and as not restricting the scope of the invention.

### DESCRIPTION OF THE FIGURES

FIG. 1: Table 1

FIG. 2: Table 2

FIG. 3: Diagram of apoptosis potentiation via CD16.

FIG. 4:
A: Potentiated apoptosis of Daudi cells by an anti-human IgG antibody.
B: Potentiated apoptosis of B cells by an anti-human IgG antibody. Apoptosis of B cells purified from peripheral blood induced by anti-HLA-DR-EMABling® antibodies (DR YB2/0) and anti-HLA DR CHO (DR CHO) antibodies at 24 h with or without the presence of a crosslinker (F(ab′)2 anti-Fc fragment of human Ig).

FIG. 5:
A: Potentiated apoptosis of Daudi cells by CD16 Jurkat
B: Potentiated apoptosis of B cells by Jurkat CD16. Apoptosis of B cells purified from peripheral blood, induced by anti-HLA-DR EMABling®t antibodies (DR YB2/0) and anti-HLA DR CHO antibodies (DR CHO) at 24 h with the presence of transfected Jurkat cells (CD 16 Jurkat) or without (WT Jurkat) by the CD16 molecule.

### EXAMPLES

### Antibodies

An anti-HLA-DR antibody was used to design a study model. It is a recombinant antibody in which the variable domains are of murine origin, whereas the constant part is of human origin.

The same sequence was expressed in the CHO line and in the YB2/0 line (these antibodies expressed in YB2/0 are called “EMABling® antibodies”).

### Cells

The clone E6.1 Jurkat cells came from the European Collection of Cell culture—ECACC). These same cells were transfected by an expression vector coding for the gamma chain (7) and by a vector coding for FcγRIIIa (L48F 158 haplotypes). These cells transfected with a DNA molecule in order to express CD16 cannot induce cytotoxicity via an ADCC mechanism.

The Daudi cells came from the American Type Culture Collection—ATCC).

The NK cells (Natural Killers) are purified from samples of peripheral blood by negative selection on Myltenyi magnetic beads.

### Induction of Apoptosis

Condition 1: the Daudi cells (2.5×10⁵ cells) are incubated with the anti-HLA-DRs (1 μg/ml) in 24-well plates (P24) for 24 h at 37° C.

Condition 2: the Daudi cells (2.5×10⁵ cells) are incubated with the anti-HLA-DRs (1 μg/ml) in the presence of an anti-human IgG (20 μg/ml) in 24-well plates (P24) for 24 h at 37° C.

Condition 3: the Daudi cells (2.5×10⁵ cells) are incubated with the anti-HLA-DRs (1 μg/ml) in the presence of Jurkat cells (2.5×10⁵ cells) in 24-well plates (P24) for 24 h at 37° C.

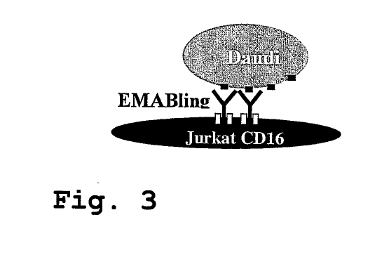

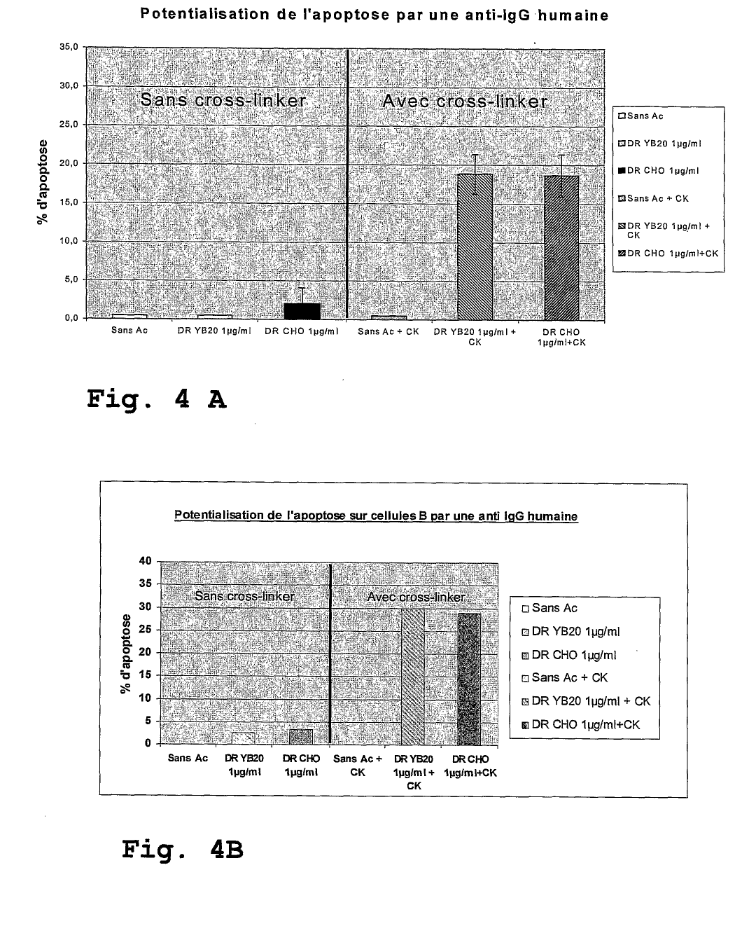

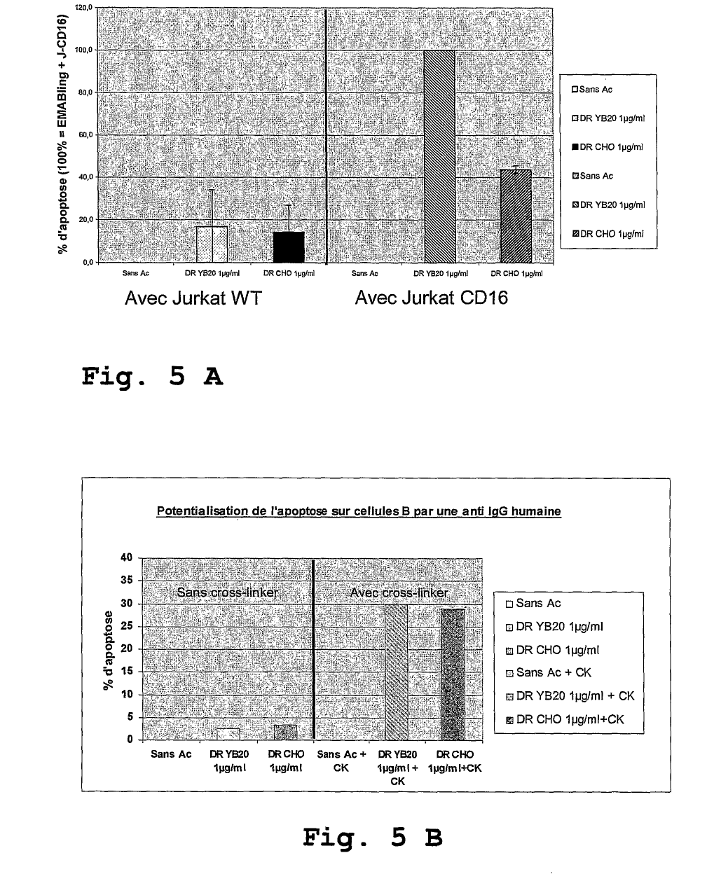

Condition 4: the Daudi cells (2.5×10⁵ cells) are incubated with the anti-HLA-DRs (1 μg/ml) in the presence of Jurkat cells transfected with CD16 (2.5×10⁵) in 24-well plates (P24) for 24 h at 37° C. (FIG. 3).

Condition 5: the Daudi cells (2.5×10⁵ cells) are incubated with the anti-HLA-DRs (1 μg/ml) in the presence of NK cells (2.5×10⁵ cells) and a mixture of 4 mM EGTA/2 mM MgCl₂, in 24-well plates (P24) for 24 h at 37° C.

### Measurement of Apoptosis Using the Annexin V/PI Technique

The cells are then harvested, washed twice then incubated with Annexin V-FITC and propidium iodide (PI) following the manufacturer's instructions (BD Biosciences). The cells are analyzed by flow cytometry (EPICS XL cytometer, Beckman Coulter) using Expo 32 software from Beckman Coulter. The binding of Annexin V corresponding to an early signal of apoptosis whereas the binding of PI comes later (DNA fragmentation), the percentage of so-called apoptotic cells is defined arbitrarily as the sum of Annexin V positive cells and doubly Aimexin V/PI positive cells.

### Example 1

### Potentiation of Apoptosis by an Anti-Human IgG Antibody (Crosslinker)

The apoptosis of Daudi cells observed in the absence of antibodies (natural death) is in the order of 10%. This basic value is inferred from the results obtained during different experiments and is therefore arbitrarily set at 0%.

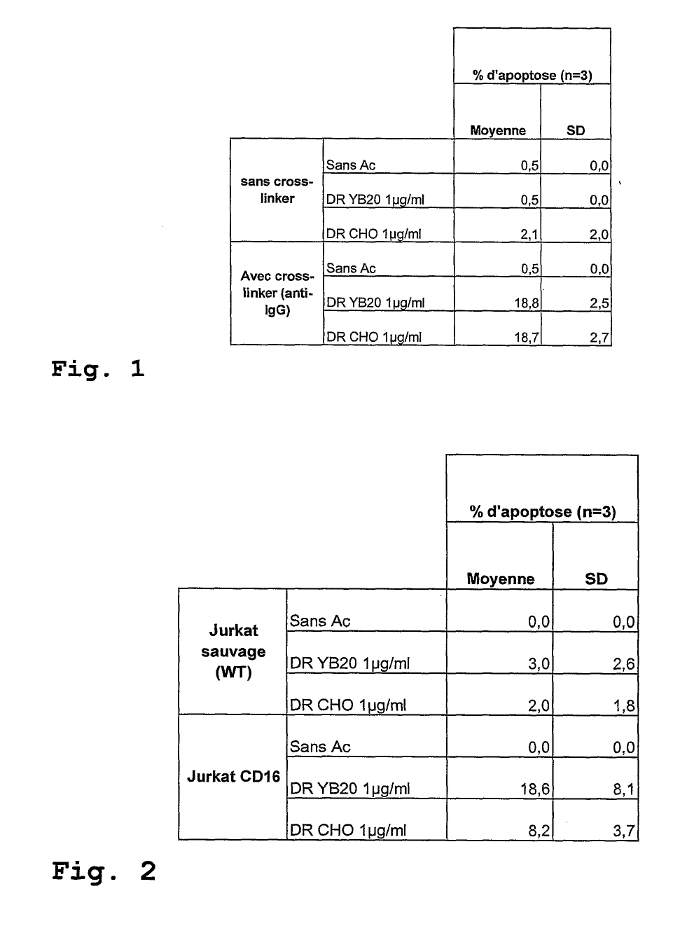

In Table 1 (cf. FIG. 1) the results are expressed as a percentage of apoptotic cells (Annexin V and PI positive). The anti-HLA-DR EMABling® and CHO antibodies used alone (condition 1) at a concentration of 1 μg/ml induce little apoptosis on Daudi (<3%). On the other hand, in the presence of an antibody directed against the Fc region of a human immunoglobulin (condition 2) which clusters the anti-HLA-DR antibodies on the surface of the Daudi cell, apoptosis is potentiated in identical manner for both antibodies increasing from less than 3% to 19%.

In FIG. 4A the results are expressed as a percentage of observed apoptotic cells, the EMABling® antibody arbitrarily representing 100% for each condition studied. Under these conditions, the statistical study shows that there are no significant differences between potentiation of apoptosis induced by the antibody expressed in CHO and by the anti-HLA-DR EMABling® antibody (p=0.85).

FIG. 4B indicating the percentage of apoptotic cells shows that the apoptosis of B cells derived from peripheral blood is only obtained in the presence of an anti-human IgG antibody. Under these conditions, this study shows that there are no significant differences between potentiation of apoptosis induced by the antibody expressed in CHO (DR CHO) and by the anti-HLA-DR EMABling® antibody (DR YB2/0).

### Example 2

### Potentiation of Apoptosis by CD16 Jurkat

The results on Daudi cells (Table 2, cf. FIG. 2) are expressed as a percentage of apoptotic cells (Annexin V and PI positive). The anti-HLA-DR EMABling® and CHO antibodies used at a concentration of 1 μg/ml in the presence of control Jurkat cells (non-transfected) respectively induce 3% and 2% apoptosis on Daudi (condition 3). In the presence of the CD16 Jurkat cell (condition 4), which may interact via CD16 with the Fc part of the anti-HLA-DR antibodies fixed to the surface of the Daudi cell, apoptosis is potentiated. In the presence of the antibodies produced by the CHO and EMABling®& cells, the final apoptosis percentage is respectively 8% and 19%. Therefore, potentiation of apoptosis in the presence of the EMABling® antibody is around 100% greater compared with that induced by the antibody produced in CHO. In FIG. 5A, the results are expressed as a percentage of observed apoptotic cells, the EMABling®& antibody arbitrarily representing 100% for each condition examined. Under these conditions, the statistical study shows that the difference in potentiation of apoptosis is very significant between the two antibodies (p<0.0001) in favour of the EMABling® antibody.

Also, apoptosis of B cells derived from peripheral blood is examined after substituting the anti-human IgG antibody (crosslinker) by a Jurkat cell transfected with CD16. The wild-type non-transfected Jurkat cells (WT) are used as negative control. The results expressed as a percentage of apoptotic cells (FIG. 5B) indicated that the apoptosis induced by anti-HLA-DR EMABling® (DR YB2/0) is strongly potentiated compared with that induced by the antibody produced in CHO (DR CHO). This property is probably due to the better fixing of anti-HLA-DR EMABling® (DR-YB2/0) on CD16 compared with anti HL-DR CHO (DR CHO).

The strong interaction of the EMABling® antibodies with CD 16 translates not only as an increase in ADCC and in cytokine secretion (cf. document EP 1 537 419) but also by potentiation of apoptosis. This imparts a major additional, functional advantage to the EMABling® antibodies, compared with the same antibody produced in CHO.

### REFERENCES

Alamartine E, Sabido O, Dumollard J M, Berthoux F, Lack of evidence for natural cytotoxicity deficiency against human ex vivo tumour cells in allograft recipients, Nephrol Dial Transplant. 1997 May; 12(5):988-94.
Anderson J L, Carlquist J F, Hammond E H. Deficient natural killer cell activity in patients with idiopathic dilated cardiomyopathy. Lancet, 1982; November 20; 2(8308):1124-7.
Clementi R, Locatelli F, Dupre L, Garaventa A, Emmi L, Bregni M, Cefalo G, Moretta A, Danesino C, Comis M, Pession A, Ramenghi U, Maccario R, Arico M, Roncarolo M G. A proportion of patients with lymphoma may harbor mutations of the perforin gene. Blood. 2005 Jun. 1; 105(11):4424-8.
Farag S S, Flinn I W, Modali R, Lehman T A, Young D, Byrd J C. Blood. 2004 Feb. 15; 103(4):1472-4. Epub2003. Fc gamma RIIIa and Fc gamma RIIa polymorphisms do not predict response to rituximab in B-cell chronic lymphocytic leukaemia.
Foa R, Fierro M T, Lusso P, Raspadori D, Ferrando M L, Matera L, Malavasi F, Lauria F. Reduced natural killer T-cells in B-cell chronic lymphocytic leukaemia identified by three monoclonal antibodies: Leu-11, A10, AB8.28. Br J. Haematol. 1986 January; 62(1):151-4.
Ghetie M A, Bright H. Vitetta E S. Homodimers but not monomers of Rituxan (chimeric anti-CD20) induce apoptosis in human B-lymphoma cells and synergize with a chemotherapeutic agent and an immunotoxin. Blood. 2001 Mar. 1; 97(5): 1392-8.
Green M R, Kennell A S, Larche M J, Seifert M H, Isenberg D A, Salaman M R. Natural killer cell activity in families of patients with systemic lupus erythematosus: demonstration of a killing defect in patients. Clin Exp Immunol. 2005 July; 141(1): 165-73.
Katrinakis G, Kyriakou D, Papadaki H, Kalokyri I, Markidou F, Eliopoulos G D.

Defective natural killer cell activity in B-cell chronic lymphocytic leukaemia is associated with impaired release of natural killer cytotoxic factor(s) but not of tumour necrosis factor-alpha. Acta Haematol. 1996; 96(1): 16-23.
Liu C, DeNardo G, Tobin E, DeNardo S. Antilymphoma effects of anti-HLA-DR and CD20 monoclonal antibodies (Lym-1 and Rituximab) on human lymphoma cells. Cancer Biother Radiopharm. 2004 October; 19(5)-545-61.
Nasrallah A G, Miale T D. Decreased natural killer cell activity in children with untreated acute leukaemia. Natural killer cell activity was evaluated in children with acute lymphocytic and acute myelogenous leukaemia. Cancer Res. 1983 November; 43(11):5580-5.
Nurmi T, Huttunen K, Lassila O, Henttonen M, Sakkinen A, Linna S L, Tiilikainen A. Natural killer cell function in trisomy-21 (Down's syndrome). Clin Exp Immunol. 1982 March; 47(3):735-41.
Portales P, Reynes J, Pinet V, Rouzier-Panis R, Baillat V, Clot J, Corbeau P. Interferon-alpha restores HIV-induced alteration of natural killer cell perforin expression in vivo. AIDS. 2003 Mar. 7; 17(4):495-504.
Takeuchi M, Nagai S, Nakajima A, Shinya M, Tsukano C, Asada H, Yoshikawa K, Yoshimura M, Izumi T. Inhibition of lung natural killer cell activity by smoking: the role of alveolar macrophages. Respiration. 2001; 68(3):262-7.
Trentin L, Zambello R, Agostini C, Ambrosetti A, Chisesi T, Raimondi R, Bulian P, Pizzolo G, Semenzato G. Mechanisms accounting for the defective natural killer activity in patients with hairy cell leukaemia. Blood. 1990 Apr. 1; 75(7): 1525-30.
Van der Kolk L E, de Haas M, Grillo-Lopez A J, Baars J W, van Oers M H. Analysis of CD20-dependent cellular cytotoxicity by G-CSF-stimulated neutrophils. Leukaemia. 2002 April; 16(4):693-9.
Ziegler H W, Kay N E, Zarling J M. Deficiency of natural killer cell activity in patients with chronic lymphocytic leukaemia. Int J. Cancer. 1981 Mar. 15; 27(3):321-7.

1. Method to induce in vitro apoptosis of a target cell by a composition of monoclonal antibodies, comprising the contacting of said antibody composition with said target cell, in the presence of cells expressing CD16 on their surface, said antibodies being directed against said target cell, and said antibody composition having a fucose content of less than 65%.

2. Method according to claim 1, characterized in that said fucose content is less than 30%.

3. Method according to anyone of claims 1 or 2, characterized in that said fucose content lies between 20% and 45%, or between 25% and 40%.

4. Method according to any of the preceding claims, characterized in that
said antibody composition is produced by the YB2/0 cell line.

5. Use of the YB2/0 cell line to produce antibodies with strong apoptosis capability.

6. Method to select antibody compositions with strong apoptosis capability comprising the following steps:
i) contacting the antibody to be evaluated with the antigen of said antibody (or a cell expressing this antigen) in the presence of cells expressing CD16 on their surface.
ii) measuring induced apoptosis.
iii) selecting antibody compositions showing induced apoptosis greater by at least 20%, even greater by 50% or more, in the presence of the cell expressing CD 16, compared with the negative control (absence of CD16).

7. Method according to claim 6, characterized in that the selected antibodies are anti-idiotype antibodies to anti-factor VIII inhibitors, antibodies directed against autoantibodies and antibodies directed against viral and/or bacterial proteins expressed on the surface of infected cells.

8. Use of a composition of monoclonal antibodies, which composition has a fucose content of less than 65%, or is obtained following the method according to anyone of claims 6 or 7, for the preparation of a medicament which, without involving the cytotoxic functions of the immune effector cells, is intended to treat a quantitative or qualitative NK-cell deficiency (no ADCC).

9. Use according to claim 8, wherein the fucose content of said composition of monoclonal antibodies is less than 30%.

10. Use according to claim 8, wherein the fucose content of said composition of monoclonal antibodies lies between 20% and 45%, or between 25% and 40%.

11. Use according to claim 8, wherein said composition of monoclonal antibodies is produced by the YB2/0 cell line.

12. Use according to any of claims 8 to 11, for the preparation of a medicament intended to treat B-CLL.

13. Use according to any of claims 8 to 11, for the preparation of a medicament intended to treat lupus erythematosus, dilated cardiomyopathy, histiocytosis with haemophagocytosis, as well as HIV and haemophilia A or B.
